(19) United States
(12) Patent Application Publication
(10) Pub. No.: US 2022/0249335 A1
(43) Pub. Date: Aug. 11, 2022

(54) COMPOSITIONS FOR IMPARTING COLOR AND TONE TO THE HAIR

(71) Applicant: L'Oreal, Paris (FR)

(72) Inventors: Emma HOWES, Lake Hiawatha, NJ (US); Melanie CRIM, Piscataway, NJ (US); Manav A. LALWANI, Secaucus, NJ (US); Heather SMITH, Clark, NJ (US)

(21) Appl. No.: 17/665,053

(22) Filed: Feb. 4, 2022

Related U.S. Application Data
(60) Provisional application No. 63/146,441, filed on Feb. 5, 2021.

(30) Foreign Application Priority Data
Jul. 28, 2021 (FR) 2108214

(51) Int. Cl. A61K 8/04; A61K 8/41; A61K 8/34; A61K 8/42

(52) U.S. Cl. CPC A61K 8/046; A61K 8/416; A61K 8/342; A61K 2800/596; A61K 2800/4322; A61K 2800/87; A61K 8/42

(57) ABSTRACT

The disclosure relates to foaming hair compositions, kits and devices comprising the compositions, and methods of use thereof. The foaming hair compositions comprise an emulsifier system, at least one amphoteric surfactant, quaternium-80, at least one direct dye or pigment, and water.

### CROSS REFERENCE TO RELATED APPLICATIONS

This application claims priority to U.S. Application 63/146,441, filed on Feb. 5, 2021, and FR2108214, filed on Jul. 28, 2021, both of which are incorporated by reference herein.

### TECHNICAL FIELD

The present disclosure generally relates to compositions and methods for imparting color and/or tone to the hair.

### BACKGROUND

It is known that consumers desire to use cosmetic and care compositions that enhance the appearance of keratinous substrates such as hair, e.g., by changing the color of hair. The process of changing the color of hair can involve depositing an artificial color onto the hair which provides a different shade or color to the hair, and/or lifting the color of the hair, such as lightening the color of dark hair to lighter shades. However, the process of lightening hair can impart a brassy tone (i.e. orange and/or yellow tones or undertones) to hair or expose brassy undertones already present in hair. In addition, undesirable brassy tones may be naturally more present and/or visible with some hair colors, or may become exposed through environmental factors such as sunlight.

Hair toner is any semi- or demi-permanent hair color that cools, tones, enhances hair tone, or imparts anti-yellowing benefits to hair. A variety of hair products can be used as toners, like tinted shampoos, conditioners, hair masks, glosses, mousses and other foam-based products, or any other product that can contain pigments that can counteract brassy tones, such as purple or blue pigments. Toners can be used on any hair color, from blonde to red, brunette and black hair to tone hair to cooler shades and help counteract warm tones.

Yet toners can also present different drawbacks and/or challenges, such as, for example, uneven application when the particular composition is applied to hair, resulting in pigment heavy streaks of hair and/or uneven coverage of brassy hair. The hair products can also weight hair down and impart other undesirable cosmetic attributes.

As such, there is a need for hair cosmetic and hair color toning compositions, including foaming compositions, that can be easily applied to and distributed throughout hair, provide anti-yellowing benefits to hair, and provide good cosmetic properties.

It has now surprisingly been found that compositions according to the disclosure can be made in a leave-in format using an emulsification system with direct dyes to provide instant toning effect without leaving the hair weighed down. These foam compositions can be easy to apply and distribute, can provide more even anti-yellowing benefits and color deposition to dyed or bleached hair, and/or can impart various advantageous sensory properties to the hair, such as softness and lightweight conditioning.

### SUMMARY

The disclosure relates to hair cosmetic and hair care compositions which comprise a toner that can be used to tone or provide anti-yellowing benefits to hair. The disclosure also relates to packaging systems comprising the compositions, and methods of using the compositions.

In various embodiments, the disclosure relates to a foaming composition comprising: (a) an emulsifier system comprising: (i) behentrimonium chloride; (ii) at least one fatty alcohol; and (iii) polyquaternium-37; (b) at least one amphoteric surfactant chosen from (C8-C20)alkylbetaines, (C8-C20)alkylamido(C1-C6)alkylbetaines, or mixtures thereof; (c) quaternium-80 present in an amount of about 0.1% to about 1% by weight, relative to the total weight of the composition; (d) at least one direct dye or pigment; and (e) water.

In some embodiments the composition is an aerosolized toning foam mousse. Thus, the composition may further comprise at least one propellant.

In an embodiment, the behentrimonium chloride is present in an amount ranging from about 0.1% to about 1% by weight, relative to the total weight of the composition. In some embodiments, the at least one fatty alcohol is chosen from cetyl alcohol, stearyl alcohol, or cetearyl alcohol, or mixtures thereof. For example, the at least one fatty alcohol can comprise, consist essentially of, or consist of cetearyl alcohol. The at least one fatty alcohol may, in various embodiments, be present in an amount ranging from about 0.1% to about 1.5% by weight, relative to the total weight of the composition.

In certain embodiments, the polyquaternium-37 is present in an amount ranging from about 0.1% to about 1% by weight, relative to the total weight of the composition.

In some embodiments, the at least one amphoteric surfactant is chosen from cocamidopropyl betaine, coco-betaine, or mixtures thereof. For example, the at least one amphoteric surfactant can comprise, consist essentially of, or consist of cocamidopropyl betaine. The at least one amphoteric surfactant may, in various embodiments, be present in an amount ranging from about 0.5% to about 2.5% by weight, relative to the total weight of the composition.

The compositions comprise Quaternium-80, which may, in various embodiments, be present in an amount ranging from about 0.1% to about 2% by weight, relative to the total weight of the composition. In certain embodiments, the composition further comprises one or more additional conditioning agents, e.g. polyester-11. The additional conditioning agents, e.g. polyester-11, may, in various embodiments, be is present in an amount ranging from about 0.01% to about 10% by weight, such as about 0.1% to about 8%, about 0.1% to about 6%, about 0.1% to about 5%, about 0.1% to about 4%, about 0.1% to about 3%, about 0.1% to about 2%, or about 0.1% to about 1% by weight, relative to the total weight of the composition.

In various embodiments, the at least one direct dye comprises External Violet 2, Yellow 5, Blue 1, Green 5, Yellow 10, or mixtures thereof. For example, the at least one direct dye comprises External Violet 2. The total amount of the at least one direct dye or pigment may, in various embodiments, be present in an amount ranging from about 0.03% to about 0.05% by weight, relative to the total weight of the composition.

In some embodiments, the composition further comprises at least one fragrance.

In various embodiments, the disclosure also relates to a method for toning the color of the hair, the method comprising applying to the hair the disclosed foaming composition.

In various embodiments, the disclosure also relates to a kit comprising a bisphenol A-free liner comprising polyamide imide and epoxy phenolic resin; where the liner surrounds the disclosed foaming composition. The disclosure also relates to an aerosol device comprising a bisphenol A-free liner comprising polyamide imide and epoxy phenolic resin; where the liner surrounds the disclosed foaming composition.

### DETAILED DESCRIPTION

The disclosure relates to compositions for caring for hair and for toning the color of hair, packaging systems comprising the compositions, and methods of using the compositions.

Toning is the neutralization of brassy hair, or the reduction or minimization of a yellow hue on the hair, e.g. after bleaching or highlighting hair or of hair of a blonde shade. The hair cosmetic compositions according to the disclosure can be used to tone or provide anti-yellowing benefits to hair.

I. Compositions

In exemplary and non-limiting embodiments, compositions according to the disclosure include an toning mousse composition, e.g. an aerosolized toning mousse composition, comprising: (a) an emulsifier system comprising: (i) behentrimonium chloride; (ii) at least one fatty alcohol; and (iii) polyquaternium-37; (b) at least one amphoteric surfactant chosen from (C8-C20)alkylbetaines, (C8-C20)alkylamido(C1-C6)alkylbetaines, or mixtures thereof; (c) quaternium-80, preferably present in an amount of about 0.1% to about 1% by weight, relative to the total weight of the composition; (d) at least one direct dye or pigment; and (e) water. The compositions may optionally further comprise additional components useful in toning mousse compositions, e.g. at least one propellant, at least one fragrance, or mixtures thereof.

Emulsifier System

The disclosed compositions comprise an emulsifier system comprising: (i) behentrimonium chloride (BETAC); (ii) at least one fatty alcohol; and (iii) Polyquaternium-37.

In various exemplary embodiments, the total amount of the behentrimonium chloride (BETAC) may vary, but typically ranges from about 0.01% to about 5%, such as from about 0.05% to about 3%, from about 0.1% to about 1%, from about 0.3% to about 0.8%, or from about 0.4% to about 0.6% by weight, relative to the total weight of the composition, including all subranges therebetween.

Compositions according to the disclosure comprise at least one fatty alcohol. As used herein, “fatty alcohol” refers to any alcohol with a carbon chain of C12 or greater, such as, for example, C12 to C22. The at least one fatty alcohol may be chosen from, for example, arachidyl alcohol, behenyl alcohol, caprylic alcohol, cetearyl alcohol, cetyl alcohol, coconut alcohol, decyl alcohol, hydrogenated tallow alcohol, jojoba alcohol, lauryl alcohol, myristyl alcohol, oleyl alcohol, palm alcohol, palm kernel alcohol, stearyl alcohol, tallow alcohol, tridecyl alcohol, or mixtures thereof. For example, in one exemplary embodiment, the at least one fatty alcohol is chosen from cetearyl alcohol, cetyl alcohol, stearyl alcohol, or mixtures thereof.

In various exemplary embodiments, the total amount of the at least one fatty alcohol may vary, but typically is at most about 5% by weight, such as from about 0.01% to about 5%, from about 0.05% to about 3%, from about 0.1% to about 1%, from about 0.3% to about 0.8%, or from about 0.4% to about 0.6% by weight, relative to the total weight of the composition, including all subranges therebetween; or ranges from 0.1% to about 1.5%, about 0.2% to about 1% or about 0.3% to about 0.8% by weight, relative to the total weight of the composition, including all subranges therebetween. In an embodiment, the disclosed compositions may comprise about 0.3% to about 0.8%, or preferably about 0.4% to about 0.7% or more preferably, about 0.4% to about 0.6%, or about 0.5%, by weight of the at least one fatty alcohol, relative to the total weight of the composition.

Polyquaternium-37 may be commercially available from BASF under the tradename of SALCARE SC 96 (comprising Polyquaternium-37 (and) Propylene Glycol Dicaprylate/Dicaprate (and) PPG-1 Trideceth-6).

In various exemplary embodiments, the total amount of the polyquaternium-37 may vary, but typically ranges from about 0.01% to about 5%, such as from about 0.05% to about 3%, from about 0.1% to about 1%, from about 0.3% to about 0.8%, or from about 0.4% to about 0.6% by weight, relative to the total weight of the composition, including all subranges therebetween.

Optionally, the amounts of (i) behentrimonium chloride; (ii) at least one fatty alcohol; and (iii) Polyquaternium-37 in the emulsifier system may be chosen to provide a weight ratio of (i) behentrimonium chloride:(ii) at least one fatty alcohol, or (i) behentrimonium chloride:(ii) at least one fatty alcohol:(iii) Polyquaternium-37 that provides optimal benefits. For example, in some embodiments, the weight ratio of (i) behentrimonium chloride:(ii) at least one fatty alcohol may range from about 2:1 to about 1:2, for example may range from about 1.5:1 to about 1:1.5, from about 1.4:1 to about 1:1.4, from about 1.3:1 to about 1:1.3, from about 1.2:1 to about 1:1.2, or may be about 1:1. In other embodiments, the weight ratio of (i) behentrimonium chloride:(i) at least one fatty alcohol:(i) Polyquaternium-37 may be about 1:1:0.85.

Amphoteric Surfactants

Compositions according to the disclosure comprise at least one amphoteric surfactant chosen from (C8-C20)alkylbetaines, (C8-C20)alkylamido(C1-C6)alkylbetaines, or mixtures thereof.

In certain exemplary embodiments, the amphoteric surfactants are chosen from (C8-C20)alkylbetaines such as the one known under the INCI names coco-betaine, (C8-C20)alkylamido(C1-C6)alkylbetaines such as the one known under the INCI name cocamidopropylbetaine, and mixtures thereof. In one embodiment, the amphoteric surfactant comprises cocamidopropylbetaine and coco-betaine. In one embodiment, the amphoteric surfactant is cocamidopropylbetaine. In one embodiment, the amphoteric surfactant is coco-betaine.

In various exemplary embodiments, the total amount of the at least one amphoteric surfactant may vary, but typically ranges from about 0.1% to about 5%, for example from about 0.5% to about 2.5%, or about 0.75% to about 1.75% by weight, relative to the total weight of the composition, including all subranges therebetween.

Conditioning Agents

The compositions according to the disclosure include quaternium-80 which also known as a conditioning agent. Quaternium-80 may be commercially available under the tradename of ABIL QUAT 3272, from the company Evonik Goldschmidt or the tradename of AEC Quaternium-80. From the company A & E Connock (Perfumery & Cosmetics).

In various exemplary embodiments, the total amount of the Quaternium-80 may vary, but typically ranges from about 0.1% to about 2%, such as from about 0.15% to about 0.8%, or about 0.2% to about 0.5% by weight, relative to the total weight of the composition, including all subranges therebetween.

Compositions according to the disclosure may further comprise at least one conditioning agent other than Quaternium-80. In an embodiment, the at least one conditioning agent other than quaternium-80 is polyester-11. In various exemplary embodiments, the total amount of additional conditioning agents may vary, but typically ranges from about 0.01% to about 5%, such as from about 0.05% to about 3%, or from about 0.1% to about 1% by weight, relative to the total weight of the composition, including all subranges therebetween.

Direct Dye

Compositions according to the disclosure may comprise at least one direct dye or pigment. In certain embodiments, the direct dyes or pigments are blue or purple to counteract the yellow, orange and/or brassy colors in hair.

Non-limiting examples of direct dyes that can be used in the disclosed compositions include direct Purple 35, Basic Blue 3 (CI 51004), BLU00100, External Violet 2 (RM 54262, CI 60730), Yellow 5 (RM 1292, CI 19140]), Blue 1 (RM 1293, CI 42090), Green 5 (RM 1294, CI 61570), or Yellow 10 (RM 900 A, CI 47005). In one exemplary embodiment, the at least one direct dye can be External Violet 2.

In various exemplary embodiments, the total amount of the at least one direct dye or pigment may vary, but typically ranges from about 0.01% to about 0.5%, such as from about 0.02% to about 0.1%, or from about 0.03% to about 0.05% by weight, relative to the total weight of the composition, including all subranges therebetween. In an embodiment, the total amount of the at least one direct dye is about 0.0442%.

Water and Other Solvents

Compositions according to the disclosure may comprise at least one solvent. The at least one solvent may be chosen from water, non-aqueous solvents, or mixtures thereof.

In some embodiments, the solvent comprises, consists essentially of, or consists of water.

In certain embodiments, the composition comprises one or more non-aqueous solvents. For example, glycerin, C₁₋₄ alcohols, organic solvents, fatty alcohols, fatty ethers, fatty esters, polyols, glycols, vegetable oils, mineral oils, liposomes, laminar lipid materials, or any a mixture thereof. Non-limiting examples of solvents which may be used include alkane polyols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

The total amount of water and/or other solvents in the compositions may vary depending on the type of composition and the desired consistency, viscosity, etc.

The solvent may be present in the composition in an amount ranging from about 60% to about 98% by weight, relative to the total weight of the composition, including all ranges and subranges therebetween. For example, in one embodiment, the total amount of solvent may be about 65% to about 95%, about 65% to about 90%, about 70% to about 95%, about 70% to about 90%, about 75% to 95%, or about 75% to 90% by weight, relative to the total weight of the composition. In certain embodiments, the solvent is primarily comprised of water, such as from about 90% to about 99%, or about 95% to about 99%, of the total solvent.

Additional Components

Compositions according to the disclosure may comprise additional components, including but not limited to fragrance, active agents, preservatives, and auxiliary components.

### Active Agents

In various embodiments, compositions according to the disclosure may optionally comprise at least one active agent such as an acid or sodium hydroxide, or mixture thereof, to provide optimized strengthening benefits to the hair. Non-limiting examples of useful acids include glycolic acid, lactic acid, malic acid, tartaric acid, citric acid, ascorbic acid, mandelic acid, azelaic acid, glyceric acid, tartronic acid, gluconic acid, benzylic acid, pyruvic acid, 2-hydroxybutyric acid, salicylic acid, trichloroacetic acid, or mixtures thereof.

The acids are typically non-polymeric and may have one (mono), two (di), or three (tri) carboxylic acid groups (—COOH). The non-polymeric mono-, di-, and tricarboxylic acids, and/or salts thereof, typically have a molecular weight of less than about 500 g/mol, less than about 400 g/mol, or less than about 300 g/mol.

Non-limiting examples of monocarboxylic acids include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, a salt thereof, and a mixture thereof.

Non-limiting examples of dicarboxylic acids include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, a salt thereof, or mixtures thereof.

Non-limiting examples of tricarboxylic acids include citric acid, isocitric acid, aconitric acid, propane-1,2,3-tricarboxylic acid, benzene-1,3,5-tricarboxylic acid, a salt thereof, or mixtures thereof.

The additional components may comprise at least one fragrance. The fragrance may comprise alcohol in a concentration range of, for example, about 10% to about 45%, about 15% to about 42%, about 20% to about 40%, about 25% to about 40%, based on the total weight of the fragrance.

In various embodiments, the total amount of fragrance may be about 0.1% to about 2%, about 0.2% to about 1.5%, about 0.3% to about 1%, about 0.3% to about 0.8%, or about 0.3% to 0.7%, by weight, relative to the total weight of the composition.

If present, the total amount of the one or more active agents may vary but typically ranges from about 0.0001% to about 10%, such as from about 0.0001% to about 5%, about 0.0001% to about 1%, about 0.001% to about 10%, about 0.001% to about 5%, about 0.001% to about 1% by weight, about 0.01% to about 10%, about 0.01% to about 5%, about 0.01% to about 1%, about 0.1% to about 10%, about 0.1% to about 5%, or about 0.1% to about 1% by weight, based on the total weight of the composition. For example, the total amount of the fragrance may range from about 0.0001% to about 0.5% by weight, based on the total weight of the composition.

### Preservatives

One or more preservatives may be included in the compositions described herein for treating hair. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, benzoic acid, chlorhexidine digluconate, ethylenediamine-tetraacetic acid (EDTA), potassium sorbate, and/or grapefruit seed extract, or a mixture thereof. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, Vitamin E (tocopherol), and a mixture thereof. In some cases, the hair-treatment compositions may include one or more preservatives selected from the group consisting of sodium benzoate, benzoic acid, chlorhexidine digluconate, chlorhexidine dihydrochloride, salicylic acid, phenoxyethanol, methyl paraben, or mixtures thereof.

The total amount of the one or more preservatives, when present, may vary. In some cases, the total amount of the one or more preservatives is about 0.01% to about 5%, about 0.01% to about 4%, about 0.15% to about 1%, or about 1% to about 3%, by weight, relative to the total weight of the composition.

### Auxiliary Components

Compositions according to the disclosure may optionally comprise any auxiliary component suitable for use in such compositions. Such components may include, but are not limited to, moisturizing agents, fatty substances and conditioning agents other than those previously described, thickeners, fillers, structuring agents, shine agents, antioxidants or reducing agents, penetrants, sequestrants, buffers, dispersants, plant extracts, opacifiers, sunscreen agents, vitamins, pH adjusting agents, and antistatic agents.

Optional auxiliary components, for example, may be present in an amount ranging up to about 15% by weight, relative to the total weight of the composition.

Optionally, the compositions may comprise up to about 100% biodegradable, sustainable, and/or environmentally friendly raw materials, such as up to about 99%, up to about 98%, up to about 97%, up to about 96%, or up to about 95%.

In various embodiments, the compositions may have a pH less than or equal to about 7. For example, the pH of the composition may range from about 4 to about 6, such as from about 4 to about 5, about 4.5 to about 6, about 4.5 to about 5, or from about 4.8 to about 5.8, including all ranges and subranges therebetween.

Compositions according to the disclosure are typically in the form of a foamable composition that may be stored as a liquid prior to foam generation. The term foam is generally understood to an aerosolized material that exhibits low to no flow under steady state. A foam can also be understood based on dynamic strain sweep test using a rheometer.

In various embodiments, the liquid composition may have the appearance of a clear, substantially clear, translucent or opaque liquid.

In at least certain embodiments, the compositions in liquid form may be stable, meaning that no or essentially no phase separation or significant change in pH or viscosity is seen, over a period of at least one week, such as at least one month, at least 6 weeks, at least 7 weeks, at least 8 weeks, at least 6 months, or at least one year under ambient conditions.

Compositions according to the disclosure are able to transform into a foam upon application of sufficient pressure and release from their containers.

One way in which foam density is described herein relates to the volume of air bubbles to the total volume of the foam, which includes the bubbles and the liquid carrier thereof. The foam of compositions according to the disclosure may thus comprise air bubbles in at least about 30%, such as at least about 40%, of the foam, and the density may thus be described as at least about 30% or at least about 40% v/v. In various embodiments, the foams may be creamy and/or fine-pored. In certain embodiments, the foam may be sufficiently stable such that it remains substantially foam-like as it is rubbed between the hands and/or worked into the hair. In at least some embodiments, the foam of compositions according to the disclosure may remain stable for more than 5 minutes, such as more than 10 minutes, more than 20 minutes, more than 30 minutes, more than 45 minutes, more than 60 minutes, or more than 90 minutes, without a significant change in volume and/or density.

In at least some embodiments, the composition according to the disclosure is characterized as providing a foam of good quality when the composition is aerosolized from the same container at different times over an extended period of time from initial preparation of the aerosol system, for example, after 1 week, after 2 weeks, after 3 weeks, after 4 weeks, after 5 weeks, after 6 weeks, after 7 weeks, after 8 weeks, after 6 months, or after one year under ambient conditions. Foam that is characterized as having “good quality” is typically described as not dissipating into liquid form before it is applied onto the hair.

The foam compositions according to the present disclosure are generally easy to spread throughout the hair, which facilitates product coverage and even toning effects on hair. The compositions further are able to provide care benefits, and in at least in some embodiments may provide style or shaping benefits to hair. The compositions may in some cases, provide conditioning benefits and/or are not sticky or waxy.

II. Packaging Systems

There are generally two types of foam hair cosmetic compositions, aerosol type and non-aerosol type. The aerosol type compositions typically require a propellant and the compositions are usually filled in an aerosol pressure resistant container and are dispensed from the nozzle of the container in the form of a foam. When the composition is dispensed from a pressurized aerosol container, a propellant may be used to force the composition out of the container. Suitable propellants include, but are not limited to, a liquefiable gas or a halogenated propellant. Examples of suitable propellants include dimethyl ether and hydrocarbon propellants such as propane, n-butane, iso-butane, CFCs, and CFC-replacement propellants. The propellants may be used singly or admixed. In various embodiments, the propellant(s) may be present in the composition, or may be present in the container and contained separately from the composition.

In some embodiments, the amount of propellant may range from about 10% to about 60%, such as about 15% to about 55%, about 20% to about 50%, about 25% to about 45%, about 30% to about 40%, about 35% to about 38%, by weight of the formulation. In other embodiments, the total amount of propellants may range from about 0.1% to about 15%, such as from about 0.5% to about 12%, from about 1% to about 10%, from about 3% to about 9%, or from about 5% to about 7% by weight, relative to the total weight of the composition, including all subranges therebetween.

The disclosed compositions can be packaged in any dispensing container that has a dispensing device configured to dispense its contents as an aerosol foam with or without controlled amount or doses. The propellant may be separated from the composition as in a two-compartment container. Other suitable aerosol dispensers are those characterized by the propellant being compressed air, which can be filled into the dispenser using a pump or equivalent device prior to use.

The present disclosure also relates to kits or packaging systems comprising the compositions described herein. The packaging systems comprise at least one container suitable for containing and/or applying the composition and/or transforming the compositions into a foam.

In various embodiments, a packaging system according to the present disclosure may include a dispensing container containing a composition disclosed herein. The composition contained in the dispensing container is generally transparent or semi-transparent, i.e., clear or semi-clear and in the form of a liquid.

III. Methods

The present disclosure also relates to methods for styling keratin fibers, e.g. human hair, using the compositions described herein. The methods generally comprise applying any of the disclosed compositions to the keratin fibers. According to the present disclosure, the compositions described herein are useful in toning hair or providing anti-yellowing properties to hair. The compositions described herein are also particularly useful for conditioning hair.

In various exemplary embodiments, the methods may include applying a toning-effective amount of a foam composition of the present disclosure to hair (wet, damp, or dry hair), before, during, and after grooming and/or styling and shaping the hair. The composition may be left on the hair for any period of time, such as a few hours or a few days, or until the next washing or rinsing of the hair.

In some embodiments, a composition of the disclosure may be applied to the hair and left on the hair, e.g. as a mousse for semi-permanently or temporality maintaining cooler or ashier hair color. In one embodiment, the hair is allowed to air dry after application of the composition, and is styled or shaped with no heat being applied to the hair. Styling or shaping the hair may involve the use of devices on hair such as a brush, a comb or running the fingers of the hand through the hair. In another embodiment, the methods may include styling or shaping the hair with a heating device before, during, or after the compositions are applied to the hair.

Additionally, the compositions provide a variety of desirable sensory benefits to the hair, for example, smoothness without weigh-down, detangling, moisture, and/or anti-shrinkage. As such, the compositions are useful in toning, while also caring for hair, conditioning hair, and/or imparting one or more of smoothness, and/or detangling. Accordingly, the instant disclosure encompasses methods for treating hair with the compositions of the instant disclosure. For example, the methods may also include applying the compositions to hair to boost volume or hold existing curl styles, as well as providing additional sensory benefits to the hair, e.g. conditioning.

As described herein, the composition is transformed into a foam when released from its container. Thus, the disclosure also relate to methods of transforming the compositions described herein into foams by applying pressure to the compositions and dispensing them through a nozzle.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” means “at least one” and thus includes individual components as well as mixtures/combinations. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

Throughout the disclosure, if the term “a mixture thereof,” or “a combination thereof” including variants, is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, +/−1%, +/−0.9%, +/−0.8%, +/−0.7%, +/−0.6%, +/−0.5%, +/−0.4%, +/−0.3%, +/−0.2%, +/−0.1%, +/−0.09%, +/−0.08%, +/−0.07%, +/−0.06%, +/−0.05%, +/−0.04%, +/−0.03%, +/−0.02%, or +/−0.01%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient or raw material.

All amounts given herein are relative to the amount of active material, unless otherwise indicated.

All percentages, parts and ratios herein are based upon the total weight of the compositions of the present disclosure, unless otherwise indicated.

As used herein, the terms “applying a composition onto keratin materials” and “applying a composition onto hair” and variations of these phrases are intended to mean contacting the keratin materials including hair and skin, with at least one of the compositions of the disclosure, in any manner. It may also mean contacting the keratin materials in an effective amount.

Unless otherwise indicated, all percentages herein are by weight, relative to the weight of the total composition.

As used herein, the term “conditioning” means imparting to hair fibers at least one property chosen from combability, moisture-retentivity, luster, shine, and softness. The state of conditioning can be evaluated by any means known in the art, such as, for example, measuring, and comparing, the ease of combability of the treated hair and of the untreated hair in terms of combing work, and consumer perception.

As used herein, the term “organic” means a material that is produced substantially without or essentially without the use of synthetic materials.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, the term “salts” refers to throughout the disclosure may include salts having a counter-ion such as an alkali metal, alkaline earth metal, or ammonium counterion. This list of counterions, however, is non-limiting.

As used herein, the term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “surfactants,” as well as any specifically-identified surfactants, includes salts of the surfactants even if not explicitly stated.

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “treat” (and its grammatical variations) refers to the application of the compositions of the present disclosure onto the surface of keratin materials, such as hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt %) of active materials, relative to the total weight of the composition.

### Example 1—Foaming Compositions

The following inventive and comparative foaming compositions were prepared according to the formulation set forth in Table 1 below.

TABLE 1

| | Inventive | Inventive | Comparative |
|---|---|---|---|
| | Composition | Composition | Composition |
| INCI Name | 2 | 1 | C1 |
| POLYESTER-11 | 0.55 | 0.55 | 0.55 |
| COCO-CAPRYLATE | 0.47 | 0.47 | 0.47 |
| BEHENTRIMONIUM | 0.49 | 0.49 | 0.49 |
| CHLORIDE | | | |
| POLYQUATERNIUM-37 | 0.42 | 0.42 | 0.42 |
| CETEARYL ALCOHOL | 0.49 | 0.49 | 0.49 |
| HELIANTHUS ANNUUS | 0.00001 | 0.00001 | 0.00001 |
| (SUNFLOWER) SEED OIL | | | |
| LAURETH-4 | 1.88 | 1.88 | 1.88 |
| QUATERNIUM-80 | 0.24 | 0.24 | |
| PROPYLENE GLYCOL | 0.23 | 0.23 | |
| COCAMIDOPROPYL | 1.07 | 1.07 | |
| BETAINE | | | |
| RAPESEEDAMIDOPROPYL | | | 2.35 |
| ETHYLDIMONIUM | | | |
| ETHOSULFATE | | | |
| QUATERNIUM-96 | | | 0.94 |
| PROPANE | 1.56 | 1.56 | 1.56 |
| BUTANE | 4.44 | 4.44 | 4.44 |
| PROPANEDIOL | | | 0.47 |
| DIPROPYLENE GLYCOL | | | 0.94 |
| EXT. VIOLET 2 | 0.044 | 0.042 | 0.038 |
| CITRIC ACID | 0.05 | 0.05 | 0.05 |
| HYDROXYETHYL UREA | 1.27 | 1.27 | 1.27 |
| DISODIUM EDTA | 0.09 | 0.09 | 0.09 |
| FRAGRANCE | 0.28 | 0.28 | 0.28 |
| ADDITIVES (one or more of | <0.1 | <0.1 | <0.1 |
| preservatives, oils, nonionic | | | |
| surfactants, humectants, | | | |
| vitamins, pH adjusters, | | | |
| antioxidants) | | | |
| WATER | QS to 100 | QS to 100 | QS to 100 |

The following comparative foaming composition was prepared according to the formulation set forth in Table 2 below.

TABLE 2
Comparative Composition 2
INCI Name

Water/Eau/Aqua
Butane
Hydroxyethyl Urea
Rapeseedamidopropyl Ethyldimonium Ethosulfate
Propane
Laureth-4
Quaternium-96
Hippophae Rhamnoides (Sea Buckthorn) Extract
Panthenol
Coco-Caprylate
Propanediol
Cetearyl Alcohol
Behentrimonium Chloride
Polyquaternium-37
Disodium EDTA
Dipropylene Glycol
Propylene Glycol
Iodopropynyl Butylcarbamate
Diazolidinyl Urea
Fragrance/Parfum
Polyester-11
Ext. Violet 2 (CI 60730)

Inventive Compositions 1 and 2 and Comparative Composition C1 and C2 were prepared according to the general process of adding the components of the emulsifier, and other ingredients one at a time to water while mixing in order to solubilize the ingredients, until the mixture is homogeneous. If necessary, an acid or base was added to adjust the pH.

The stability of the pH and the foam quality of the inventive composition was assessed over a period of time of storing the compositions (after initially making the composition, at week 1, at week 2, at week, 3, at week, 4, at week 5, at week 6, at week 7, and at week 8 at various temperatures—room temperature, 25° C., 40° C., and 60° C. to evaluate foam quality based on the foam collapsing or being acceptable over time.

The inventive and comparative compositions (bench) showed comparable stabilities with respect to pH and good foam quality over time (from initial up to 8 weeks). However, the inventive formulas performed better than the comparative compositions with respect to cosmeticity of the hair, including a more lightweight feel and softness, and toning (reduced/minimized brassiness or anti-yellowing effect), including better color deposition, as evident from the study below.

### Example 2—Cosmetic and Sensory Properties Evaluation

In a half head study, a comparative test was conducted to evaluate the performance of Inventive Composition 1 in comparison with either Comparative Composition C1 or the commercially available Comparative Composition C2, as described in Example 1. In this test, the hair of 4 volunteer participants was shampooed and towel dried. Similar amounts of the test compositions were each respectively applied to the hair on a designated side of a participant's head. The hair was subsequently blow-dried and styled.

The cosmetic and sensory attributes of the hair and compositions were assessed. The following improvements were demonstrated when using Inventive Composition 1 over the comparative compositions:

Better toning
Cooler effect
Lighter feel
Improved viscosity
Improved spreadability
Damp hair detangling
Dry hair smoothness
Dry hair split end seal
Dry hair color visibility

Thus, Inventive Composition 1 provided better toning properties, a cooler effect (less yellow and/or brassy hair), an evenness of toning effect on the hair, and a more evident neutralization power and ashy finish. Inventive Composition 1 also did not have an undesirable very coated (waxy) feel which was observed for Comparative Composition 1.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1. A foaming hair cosmetic composition comprising:
(a) an emulsifier system comprising:
(i) behentrimonium chloride;
(ii) at least one fatty alcohol; and
(iii) polyquaternium-37;
(b) at least one amphoteric surfactant chosen from (C8-C20)alkylbetaines, (C8-C20)alkylamido(C1-C6)alkylbetaines, or mixtures thereof;
(c) quaternium-80;
(d) at least one direct dye or pigment; and
(e) water.

2. The composition according to claim 1, wherein the behentrimonium chloride is present in an amount ranging from about 0.1% to about 1% by weight, relative to the total weight of the composition.

3. The composition according to claim 1, wherein the at least one fatty alcohol is chosen from cetyl alcohol, stearyl alcohol, or cetearyl alcohol, or mixtures thereof.

4. The composition according to claim 1, wherein the at least one fatty alcohol comprises cetearyl alcohol.

5. The composition according to claim 1, wherein the total amount of fatty alcohols ranges from about 0.1% to about 2.5% by weight, relative to the total weight of the composition.

6. The composition according to claim 1, wherein the polyquaternium-37 is present in an amount ranging from about 0.1% to about 1% by weight, relative to the total weight of the composition.

7. The composition according to claim 1, wherein the at least one amphoteric surfactant is chosen from cocamidopropyl betaine, coco-betaine, or mixtures thereof.

8. The composition according to claim 1, wherein the at least one amphoteric surfactant comprises cocamidopropyl betaine.

9. The composition according to claim 1, wherein the total amount of amphoteric surfactants ranges from about 0.5% to about 2.5% by weight, relative to the total weight of the composition.

10. The composition according to claim 1, wherein the quaternium-80 is present in an amount of about 0.1% to about 1% by weight, relative to the total weight of the composition.

11. The composition according to claim 1, further comprising at least one additional conditioning agent.

12. The composition according to claim 1, further comprising polyester-11 in an amount ranging from about 0.1% to about 3% by weight, relative to the total weight of the composition.

13. The composition according to claim 1, wherein the at least one direct dye comprises External Violet 2, Yellow 5, Blue 1, Green 5, Yellow 10, or mixtures thereof.

14. The composition according to claim 1, further comprising at least one propellant.

15. The composition according to claim 1, wherein the composition is an aerosolized toning foam mousse.

16. The composition according to claim 1, wherein the weight ratio of the total amounts of (i) behentrimonium chloride:(ii) fatty alcohols ranges from about 2:1 to about 1:2.

17. The composition according to claim 1, wherein the weight ratio of the total amounts of (i) behentrimonium chloride:(ii) fatty alcohols is about 1:1.

18. The composition according to claim 1, wherein the weight ratio of the total amounts of (i) behentrimonium chloride:(ii) fatty alcohols:(iii) polyquaternium-37 is about 1:1:0.85.

19. A method for toning hair, the method comprising applying to hair a foaming composition comprising:
(a) an emulsifier system comprising:
(i) behentrimonium chloride;
(ii) at least one fatty alcohol; and
(iii) polyquaternium-37;
(b) at least one amphoteric surfactant chosen from (C8-C20)alkylbetaines, (C8-C20)alkylamido(C1-C6)alkylbetaines, or mixtures thereof;
(c) quaternium-80;
(d) at least one direct dye or pigment;
(e) water; and
(f) optionally at least one propellant.

20. An aerosol device comprising:
(1) a bisphenol A-free liner comprising polyamide imide and epoxy phenolic resin, wherein the liner surrounds a composition comprising:
(a) an emulsifier system comprising:
(i) behentrimonium chloride;
(ii) at least one fatty alcohol; and
(iii) polyquaternium-37;
(b) at least one amphoteric surfactant chosen from (C8-C20)alkylbetaines, (C8-C20)alkylamido(C1-C6)alkylbetaines, or mixtures thereof;
(c) quaternium-80;
(d) at least one direct dye or pigment; and
(e) water; and
(2) a dispensing device configured to dispense the composition as a foam,
wherein the aerosol device, the composition, or both comprise at least one propellant.
